(19) United States
(12) Patent Application Publication
(10) Pub. No.: US 2014/0052067 A1
(43) Pub. Date: Feb. 20, 2014

(54) DEVICES FOR PUNCTURING FOR A HUMAN OR ANIMAL BODY'S MEMBRANE

(71) Applicants: Marion Sausse, Waterloo (BE); Michel Deleers, Linkebeek (BE); Denis Vandormael, Vezin (BE); Cyrille Lenders, Grez Doiceau (BE)

(72) Inventors: Marion Sausse, Waterloo (BE); Michel Deleers, Linkebeek (BE); Denis Vandormael, Vezin (BE); Cyrille Lenders, Grez Doiceau (BE)

(73) Assignee: UNIVERSITE LIBRE DE BRUXELLES, Bruxelles (BE)

(21) Appl. No.: 14/006,068

(22) PCT Filed: Mar. 14, 2012
(86) PCT No.: PCT/EP2012/054463

§ 371 Date: Oct. 30, 2013

(30) Foreign Application Priority Data
Mar. 18, 2011 (EP) 11158858.8
Jul. 15, 2011 (EP) 11174211.0

(51) Int. Cl. A61M 5/158; B29C 45/00

(52) U.S. Cl. CPC A61M 5/158; B29C 45/0053
USPC 604/173; 264/232; 264/478

(57) ABSTRACT

Device for puncturing a membrane of a human or animal body so as to introduce a liquid formulation thereinto, provided with a patch (10) made of a polymeric material, includes a base surface (12) having a distal side and a proximal side, and a plurality of projections (11), each projection projecting from the distal side of the base surface to end in a tip (13) arranged for puncturing the membrane. Each projection (11) has a cavity (14) and a shell (111) enveloping the cavity at the distal side. The cavity is open towards the proximal side of the base surface. The shells (111) have substantially constant thicknesses. The projections (11) include fluid channels (150) emanating from the cavities (14), running through the shells to end at orifices (15) provided in the shells, offset from the tips (13), the fluid channels being oriented obliquely relative to the proximal-distal axis (16, 160). A method provides for manufacturing the patch.

The present invention relates to devices for puncturing a membrane of a human or animal body, in particular for puncturing or penetrating the skin. The membrane is punctured with the aim of administering a therapeutic or cosmetic substance percutaneously. More particularly, the present invention relates to devices provided with an array of micro needles arranged for puncturing the membrane.

Drug delivery devices enabling the percutaneous administration of drugs by puncturing the skin with plurality of micrometric needles are known. These are roughly divided into systems wherein the drug is first applied onto the skin or onto the micro needles and the skin is punctured afterwards, see e.g. WO 02/49711, and systems wherein the micro needles are hollow so that a liquid formulation is injected through the needles into the skin, see e.g. U.S. Pat. No. 3,964,482, US 2005/143713.

Compared to hypodermic syringes, which deliver the drug through a single hollow needle, the transdermal delivery through a plurality of micro needles causes less pain and is less prone to misuse, so that it has the potential of being used for the self-administration of drugs. Also, the puncturing of the skin can improve the uptake of some substances which would otherwise have to be administered onto the skin only.

One difficulty arising in systems with hollow micro needles is that all the micro needles arranged in the array should puncture the skin, and preferably they should all perforate at least the stratum corneum. When not all the needles of the array puncture the skin, then part of the substance is not administered and hence the indicated dose is not attained. Those needles that do not arrive at puncturing the skin form liquid passageways with reduced flow resistance, so that a significant amount of drug may be spilt.

The force required for puncturing the skin can be significant and depends on skin type and on the part of the skin where the puncturing is performed. There exists thus a risk of damaging some needles when an excessive force needs to be applied. The damaged needles would then most likely fail to puncture the skin.

In the prior art, attempts have been made to reduce the puncturing force. It is known from WO 2010/117602 to make hollow micro needle arrays with the aid of a stacked laminate mould. A first mould half is formed by stacking a plurality of plates such that they extend perpendicular to a major mould surface which comprises micro needle cavities. The micro needle cavities are formed by providing grooves at those edges of the plates which lie in the major mould surface. The plates have a surface roughness so that submicrometre spacing between the plates is obtained, which allows for venting the cavities and obtaining a complete filling of the cavities with the injection moulding material. Micro needles with tip dimensions on the order of 1 μm are reported. The micro needles are hollow and comprise a channel developing substantially along the micro needle axis and opening on the micro needle wall.

DE 10 2008 052 749 describes micro needle arrays wherein the micro needles comprise a pedestal in the form of a truncated cone with on top an inserting end having the form of an obliquely truncated cylinder or cone. A protruding puncturing force reducing member is provided adjacent or onto the oblique surface of the inserting end. The pedestal comprises a cavity, from which a cylindrical fluid channel emanates axially, running through the inserting end, with an outlet situated in the oblique plane of truncation.

It is known from WO 2009/130926 and from WO 2010/010974 to provide the hollow micro needles on a convex surface, so that a surface drawn through the needle tips forms a convex surface as well. As a result, the puncturing force can be concentrated on only some of the needles, instead of being spread over all needles. The transdermal administration device of the above two documents further includes a system to peel off the stratum corneum prior to penetrating the skin with the micro needles. Since the stratum corneum is the toughmost layer of the epidermis, this has the advantage that penetration of the micro needles is eased and that the mechanical strength requirements on the needles may be relaxed. On the other hand however, an additional operation of skin peeling needs to be carried out, which makes the system less user-friendly and more complex.

In US 2008/183144 it is proposed to tension the skin before inserting the micro needles, so as to facilitate needle insertion. However, for some parts of the skin this will not suffice, or the amount of tensioning will not be sufficient. Particularly when developing micro needle puncturing devices meant for self administration, an easy and fail free device is required, which can cope with careless handling by a patient.

Another problem is that of cost effective production of the micro needles, particularly since micro needle assemblies are typically intended for single use and hence disposable.

In this regard, US 2010/0305516 describes to manufacture hollow micro needle arrays based on an electroforming process. Hollow micro needles are formed by metallic deposition in cavities of an embossed stacked plate comprising upper and lower plates and an intermediate separation layer. The upper and lower plates are separated and grooves are formed in the plane of separation, which intersect with the micro needles. By so doing side outlets are formed. The upper and lower plates are then joined without the separation layer and bonded under a hot press. The micro needles are then removed from the stacked plates by peeling them off or by chemical dissolving the plates. It is clear from the above that this process requires quite a number of different manufacturing steps, such that it is not as cost effective as the authors describe. Furthermore, materials for the micro needles are limited to some metals.

A much more cost effective way to manufacture micro needles is with injection moulding, which is also the way contemplated in the present invention. By way of example, documents WO 2010/117602 and DE 10 2008 052 749, both of which have already been discussed, describe to manufacture the micro needles directly by injection moulding. However, current moulds used for producing polymeric micro needles have a high complexity and a relatively short lifetime due to the small dimensions of the micro needles, and particularly of the fluid channels.

In WO 2010/117602, the male part of the mould is provided with projections which are intended to make contact with the cavities in the female mould in order to provide outlets of the fluid channel through the micro needle. It is described to provide the mould projections with an ability of self-alignment. However, self-alignment can only be obtained by making the projection suitably slender at the tip thereby providing an increased flexibility. This in fact limits the design geometry of the internal fluid channel of the micro needles and makes the mould more fragile.

In DE 10 2008 052 749, the fluid channels through the inserting ends are cylindrical, which are produced by slender inserts in the moulds. This places very high constraints on the precision of alignment of the mould halves and it can be seen that the slightest misalignment can damage the inserts. As an alternative, the document describes that the fluid channels can be made by laser drilling. This however is only a feasible option if the thickness through which to drill is sufficiently small, since otherwise the heat provided by the laser radiation will melt or soften the thermoplastic material, thereby greatly affecting the external geometry, and the inserting tip in particular. In view of the proposed micro needle geometry, the option does not seem viable.

Since moulds for injection moulding are very costly, there is a need in the art to provide an injection moulding method in which the moulds are less complex and have a longer lifetime.

Yet another issue with thermoplastic micro needles is that their strength should be sufficient to withstand the puncturing force in order to avoid collapse of the micro needle. Often this issue is resolved by providing the micro needles with sufficiently thick walls. As a result, the space available for the hollow parts, such as the fluid channel is reduced.

The latter causes problems in that the hollow micro needles suffer from relatively large flow resistances due to the long and small internal channels for fluid delivery.

It is therefore an aim of the present invention to provide devices for transdermal administration of a liquid, which overcome the above specified problems. In particular, it is an aim of the present invention to provide cost effective devices for transdermal administration of a liquid through micro needles in order to allow use of such devices on a mass scale. It is a further aim of the invention to provide solutions which overcome the drawbacks of the prior art and in particular facilitate penetration through the skin and/or fluid delivery.

According to the invention, there is hence provided a device for puncturing the membrane of a human or animal body as set out in the appended claims. The device comprises a base surface having a distal side and a proximal side, and a plurality of projections (referred to as micro needles), each projection projecting from the distal side of the base surface to end in a tip arranged for puncturing the membrane.

Each micro needle comprises an internal cavity with a cross section which varies along a proximal-distal axis (which can refer to e.g. a normal to the base surface), such as a conically shaped cavity. The cavity is enveloped by a shell which forms the micro needle wall.

According to an aspect of the invention, the wall has a substantially constant thickness, at least between the base surface and the tip. The cavity is open towards the proximal side of the base surface (towards the side of the source of liquid that is to be administered). The shell and the base surface are made of a polymeric material. An orifice is provided in the shell for delivery of fluid to the body. The orifice is arranged sideways of the tip, which allows freedom in designing the tip for optimal strength and easy penetration, and prevents or reduces clogging.

According to an aspect of the invention, the orifices form the outlets of fluid channels emanating from the cavities and running through the shells. These fluid channels are oriented obliquely relative to the proximal-distal axis.

Such a configuration has an advantage that the flow resistance through the micro needle is mainly determined by the size of the orifice and the length of the channel. Since the channel only extends through the thickness of the shell, flow resistance is significantly reduced compared to needles having cylindrical channels all along. As an important aspect of the present invention, flow resistance is made practically independent of the length of the needle.

Another advantage is that the position and orientation of the channels and orifices make it easy and cost-effective to form them only after production of the micro needles (e.g. by laser ablation), without any risk of damaging the tips of the projections and with a certainty that channels will always run fully through the shell.

According to another aspect of the invention, there is provided a disposable unit for use in devices for transdermal administration of a liquid, as set out in the appended claims. An advantage of such a disposable unit is that it comprises a tubular needle or other piercing member which protrudes proximally, hence opposite to the micro needles. The tubular needle allows piercing a cartridge, such that only a small part of a fluid delivery device is made disposable, whereas an injection device accepting the disposable unit and the cartridge can be re-used.

According to another aspect of the invention, there is provided a method of manufacturing a patch of micro needles, as set out in the appended claims. The method enables cost effective production of patches with micro needles based on injection moulding processes with inserts having reduced complexity and a comparatively longer lifetime.

### BRIEF DESCRIPTION OF THE DRAWINGS

FIGS. 1-3 represent cross sections of patches of hollow micro needles according to the invention. FIG. 1 represents micro needles projecting from a planar base surface and having substantially equal lengths. FIGS. 2 and 3 represent micro needles wherein the tips are arranged in a convex shaped surface. In FIG. 2, this is obtained by a convex base surface, whereas in FIG. 3, this is obtained by arranging needles of differing lengths.

FIG. 4 represents schematically a percutaneous injection device making use of a patch of micro needles according to the invention.

FIG. 5 represents a diffusor according to the invention, comprising a patch of micro needles.

FIG. 6 A-E represent different views of an injection device for the administration of a liquid formulation according to an aspect of the invention. FIG. 6A shows an external view. FIG. 6B shows the device of FIG. 6A partly uncovered. FIG. 6C shows an enlarged view of items 64-66 of FIG. 6B. FIGS. 6D-E show transparent plan views of the device of FIG. 6A as indicated.

FIG. 7 represents different steps in the use of the injection device of FIGS. 6A-E.

FIG. 8 represents male and female inserts of an injection mould to produce patches according to the invention.

FIG. 9 represents steps in a process of manufacturing a female insert of an injection mould to produce patches according to the invention.

FIG. 10 represents schematically identical male and female inserts of an injection mould to produce patches of micro needles according to an aspect of the invention.

FIG. 11 represents a micro needle 110 having a truncated tip in sectional view. Do is the size (diameter) of the micro needle at the base. H is micro needle height (base to tip). a is cone angle. t is shell thickness. rₜ is radius of truncated tip. L is length of channel 150. h is distance between base and orifice 15. H-h is orifice offset distance relative to the tip. β is inclination angle of channel 150 relative to axis 160.

### DETAILED DESCRIPTION OF THE INVENTION

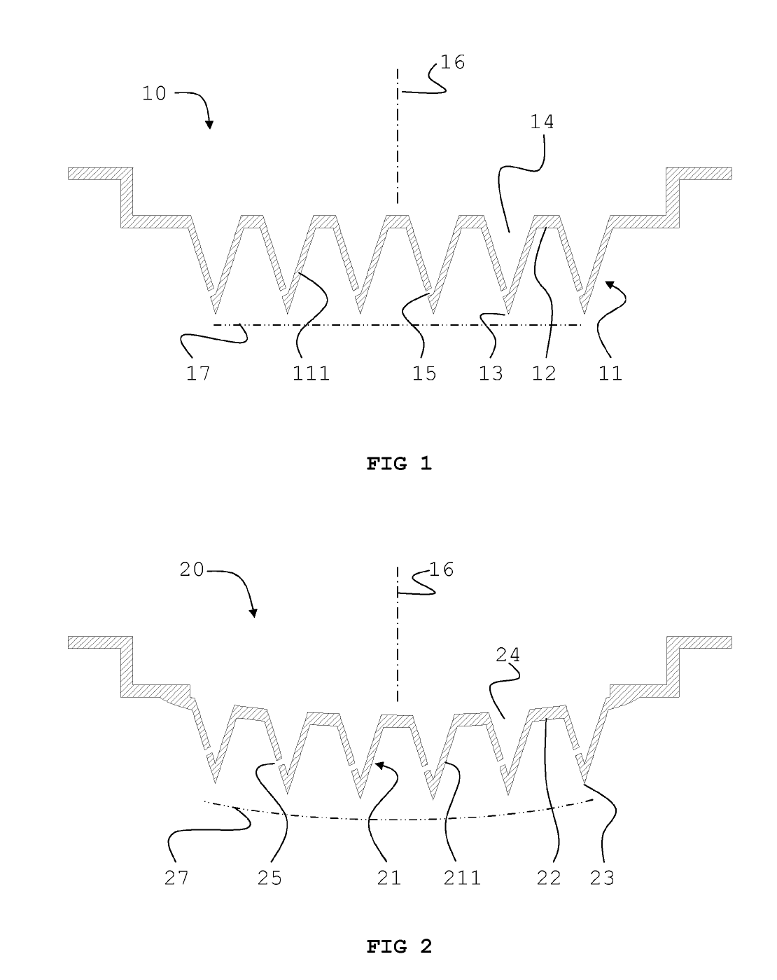

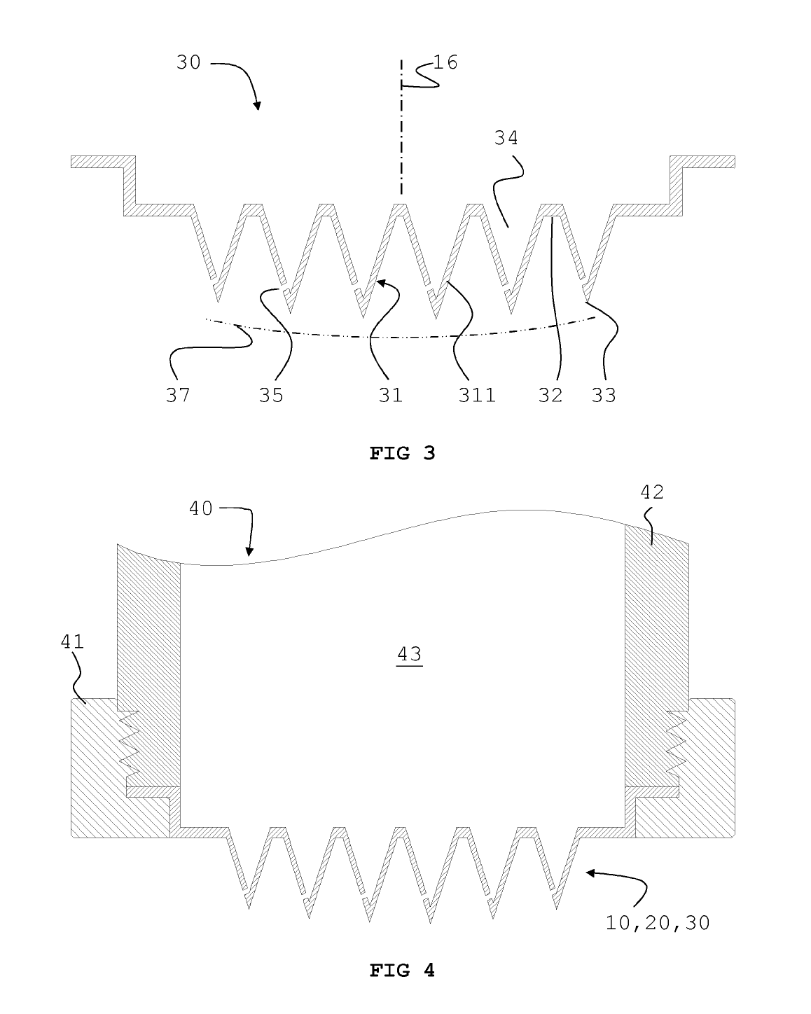

FIGS. 1 and 2 represent cross-sections of patches 10, 20 provided with projections 11, 21 according to the invention. The projections 11, 21 will be referred to as micro needles in what follows.

The micro needles 11, 21 are arranged for puncturing a biological membrane of a human or animal body. The most important membrane will be the skin, but other membranes, such as internal membranes, are envisaged as well. Referring to the skin, the expression puncturing the skin as intended in the present invention refers to penetration through at least the stratum corneum of the epidermis.

Taking the above into consideration, the terms distal side and distal direction indicate a location or direction at or towards the target membrane (which is to be punctured) . The terms proximal side and proximal direction indicate a location or direction opposite to or away from the target membrane.

The micro needles 11, 21 project or protrude from a base surface 12, 22 and terminate in a tip 13, 23. The micro needles 11, 21 and the tips 13, 23 are oriented in or along the distal direction. The tip 13, 23 should therefore be such that puncturing of the envisaged biological membrane is possible. The distance between base surface 12, 22 and tip 13, 23—i.e. the length of the needle—is inter alia dependent on the intended depth of penetration and is preferably smaller than or equal to 2000 μm, preferably smaller than or equal to 1500 μm, preferably smaller than or equal to 1000 μm in order to avoid puncturing the innerve part of the skin. Said distance (length) is preferably at least 100 μm, more preferably at least 200 μm, more preferably at least 500 μm, and most preferably at least 700 μm in order to efficiently penetrate the stratum corneum.

At the base surface, the micro needles' cross section (locally parallel to the base surface) can have an outer linear size (e.g. outer diameter) smaller than or equal to 1000 μm. The micro needles' cross section can have an outer linear size larger than or equal to 250 μm at the base surface, which size can be larger than or equal to 500 μm. At the base surface, the micro needles' cross section can have an outer linear size falling in the range between 100 μm and 600 μm, preferably between 150 μm and 600 μm, more preferably between 150 μm and 500 μm, even more preferably between 150 μm and 400 μm.

The micro needles' external geometry can have a conical shape in its broadest interpretation (i.e. with a base of any shape: polygonal (pyramid), circular, etc.). It can be of hyperboloid shape. These shapes can be truncated or rounded at the tip end. Other shapes, or a combination of the above shapes are possible as well. The micro needles' external geometry can have a shape of an advantageously right circular cone. The cone tip can be truncated or rounded.

The interdistance between adjacent micro needles 11, 21 preferably falls in the range between 500 μm and 2000 μm (measured between centre lines).

Each micro needle 11, 21 is provided with an internal cavity 14, 24 around which a shell 111, 211 is arranged. The shell 111, 211 forms the cavity's envelope or covering at the distal side and hence forms the wall of the micro needle 11, 21. The cavity 14, 24 is open towards the base surface 12, 22 and extends along at least 50% of the micro needle's length, preferably at least 55%, preferably at least 60%, preferably at least 75%.

According to the invention, the shell 111, 211 has a substantially constant thickness, in particular between the (distal side of the) base surface 12, 22 and the tip 13, 23. The shell's thickness is therefore substantially constant both in a direction along the micro needle's axis and tangentially around the centre line. For reasons of strength, the tip can have an increased thickness compared to the shell.

In the context of the present invention, a thickness will be referred to as substantially constant when thickness variations (both in the positive and in the negative sense) about an average value advantageously do not exceed 20 μm, advantageously do not exceed 15 μm, advantageously do not exceed 10 μm, advantageously do not exceed 7.5 μm, advantageously do not exceed 6 μm. Advantageously, thickness variations (both in the positive and in the negative sense) about an average value do not exceed 15% of the average value, advantageously do not exceed 10% of the average value, advantageously do not exceed 5% of the average value, notwithstanding that a minimal permissible variation can be selected among the above indicated absolute values.

The cavity 14, 24 is therefore not a channel or duct of constant cross section, but has a varying cross sectional size, advantageously reducing in size from base to tip. Hence, it is not cylindrical. The term cross section in this paragraph is to be considered in a plane perpendicular to the micro needle's axis or centre line. Hence, the micro needle's cross section varies in size along the centre line or axis of the micro needle.

The cavity 14, 24 can have a same shape as the external shape of the micro needle 11, 21. The cavity 14, 24 can have a conical shape in its broadest interpretation (i.e. with a base of any shape: polygonal (pyramid), circular, etc.). It can be of hyperboloid shape. Other shapes, or a combination of the above shapes are possible as well.

Advantageously, the shape of the cavity can be obtained by a linear translation of the external shape of the micro needle along an axis, such as the centre line or a proximal-distal axis 16. As will be explained later, this can economize manufacturing.

The tip 13, 23 forms one terminal end of the shell (distal end). At the other end, the shell terminates in the base surface 12, 22. The shell can have a thickness to provide for sufficient strength of its own to resist the puncturing force.

An advantage of micro needles with shells of constant thickness is that, when puncturing the skin, stress concentrations are avoided since they distribute evenly over the shell. This in turn allows to design micro needles with comparatively thinner shells.

The above advantage is particularly true for shells which have an axisymmetric shape, such as axisymmetric about the micro needle's centre line, in particular a centre line parallel with the proximal-distal axis 16.

It will be convenient to note that within a same patch different micro needles with different thicknesses can be provided, as long as the shells each have a constant thickness. It will however be advantageous to provide micro needles on a same patch all having a same shell thickness.

The shell's thickness can depend on the type of material used, and on the toughness of the target membrane. Advantageously, the shell has a thickness smaller than or equal to 150 μm, advantageously smaller than or equal to 120 μm, advantageously smaller than or equal to 100 μm. The thickness of the shell is advantageously larger than or equal to 10 μm, advantageously larger than or equal to 20 μm, advantageously larger than or equal to 30 μm. Preferably, the shell has a thickness falling in the range between 150 μm and 10 μm, preferably between 120 μm and 20 pm, preferably between 100 μm and 30 μm.

The shell 111, 211 is provided, on its external (distal) surface, with an (at least one) orifice 15, 25 in fluid communication with the cavity 14, 24. According to the invention, the orifice 15 constitutes the outlet of a channel 150, as shown with reference to FIG. 11, which runs through the shell 111, from cavity to orifice. The channel 150 has a length L which depends on the thickness t of the shell 111 and on the orientation of the channel relative to the shell.

A liquid formulation, which can be a medicament, a drug or any other substance for medical or cosmetic use, is administered transdermally by passing from the cavity 14, 24 through the channel 150 and orifice 15, 25.

According to an aspect of the invention, the orifice 15, 25 is not located at the tip, but sideways thereof. Preferably, the orifice's centre is offset with reference to the tip 13, 23 by at least 35 μm, preferably at least 50 μm, as measured in projection perpendicularly on the micro needle's axis. On the one hand, this allows designing the tip with more freedom to obtain optimal penetration. On the other hand, it can prevent or at least reduce clogging of the orifice 15, 25 when perforating or puncturing the membrane.

The orifice 15, 25 has a (largest) cross sectional size preferably smaller than or equal to 150 μm, preferably smaller than or equal to 120 μm, preferably smaller than or equal to 100 μm. The size of the orifice 15, 25 is preferably larger than or equal to 10 μm, preferably larger than or equal to 15 μm, preferably larger than or equal to 20 μm and can be larger than or equal to 30 μm in cross section. Said size of the orifice refers to a linear size, such as a diameter.

The channel 150 advantageously has a substantially same cross sectional size as the orifice 15. Advantageously, the size of the orifice, and the channel is substantially circular.

According to an aspect of the invention, the channel is oriented inclined relative to the axis 160 of the micro needle 110, which advantageously coincides with the proximal-distal direction. The channel 150 is advantageously oriented relative to the micro needle's axis 160 under an angle β of at least 15°, advantageously at least 25°, advantageously at least 30°. The angle β of channel orientation is advantageously smaller than or equal to 90°, advantageously smaller than 90°, advantageously smaller than or equal to 80°, advantageously smaller than or equal to 75°.

Advantageously, the channel 150 is oriented substantially perpendicularly to the shell 111, in which case its length L equals the shell's thickness t.

The orifice's channel can have a length of at least 20 μm, at least 30 μm, at least 40 μm, or even at least 50 μm, which length is at least the shell's thickness, but can be larger.

Providing orifice channels with a given length L can help in making the discharge rate among different micro needles more uniform. The size or diameter of the orifice 15, 25 and the length L of its channel can be so chosen that a good uniformity in the discharge rate among different micro needles can be obtained without increasing too much the flow resistance (and hence keeping the discharge rate—in absolute value—at an acceptable level). Indeed, by providing a cavity which is significantly larger in size than the orifice (and the channel), the dependence of flow resistance on the length or position of the needle is greatly reduced. This allows making the discharge rate of the micro needles uniform and independent of their position on the patch.

The size of the cavity 14, 24 at the base surface preferably falls in the range between 80 μm and 450 μm. The cavity's size can be larger than 450 μm. For example, the cavity can have a size smaller than or equal to 950 μm at the base surface. It can as well be smaller than or equal to 400 μm. It can be larger than or equal to 100 μm. The cavity's size refers to the largest dimension in cross section measured at the base surface.

For orifices having a size or diameter falling in the range between 40 μm and 100 μm and a channel length between 20 μm and 100 μm, improved results can be obtained in this regard.

Advantageously, the size of the orifice 15, 25 is smaller than or equal to half of the cavity's size. A preferred range for the orifice's size is between 0.1 and 0.5 times the cavity's size, with the cavity's size defined as indicated above. The orifice's size can be at least 0.15 times the cavity's size.

It will be convenient to note that in the micro needles described in WO 2010/117602, the cavities discharge directly to the orifices. These micro needles therefore do not comprise fluid channels as contemplated in the present invention. Conversely, in DE 10 2008 052 749, the fluid channels are oriented axially, parallel to the proximal-distal direction, actually discharging at the tip end.

The tip is suitably sharp to allow smooth penetration through the stratum corneum. Preferably, the tip 13, 23 has a radius of curvature smaller than or equal to 50 μm, preferably smaller than or equal to 25 μm, preferably smaller than or equal to 15 μm, preferably smaller than or equal to 10 μm.

The tip can have a radius of curvature of at least 10 μm, preferably at least 15 μm.

Larger tip radii can, in conjunction with a disposition of micro needles of different lengths as will be described, still provide a moderate to low puncturing force. An advantage however, is that micro needles with larger tip radii are more economical to produce.

Referring to FIG. 11, in case the micro needle is formed as a truncated cone, the tip is flat and the above indicated size ranges apply to the radius (rₜ) of the tip.

The tip angle (e.g. cone angle α) is preferably smaller than 60°. It can be smaller than 45°, or smaller than 40°. The cone angle is preferably at least 30°.

The micro needles 11, 21 can be arranged according to a regular pattern, such as in an array with polygonal (square, triangular, rectangular, hexagonal, etc.), circular or elliptical geometry. A patch 10, 20 can comprise at least 3, preferably at least 5, preferably at least 9 micro needles 11, 21. The number of micro needles arranged in the array (provided on the patch 10, 20) is preferably less than or equal to 100. A patch 10, 20 preferably comprises between 9 and 36 micro needles 11, 21.

Referring to FIG. 1, the base surface 12 can be planar and the micro needles 11 can have equal length. As a result, a surface that is drawn through the tips 13 is a plane 17.

FIG. 2 depicts an alternative arrangement in accordance with an aspect of the invention. According to this aspect, the micro needles 21 are so arranged that the surface 27 that can be drawn through the micro needles' tips 23 has a convex shape, i.e. is curved distally outwards. The surface 27 is convex along at least one direction and preferably convex along two orthogonal directions.

The fact that the micro needles' tips 23 are arranged in an imaginary surface 27 that is convex means that, in projection on a normal to the base surface 22, and preferably on the normal coinciding with the proximal-distal axis 16, said tips are arranged at different positions (heights) along the normal. This arrangement allows a time-shifted puncturing or penetration of the biological membrane by the different needles, in the sense that the time instant at which the micro needles 21 make first contact with and initiate penetration/puncturing into the biological membrane can be different for different micro needles. As a result, the puncturing force can be concentrated on a smaller number of needles, so that penetration is facilitated. It should however be noted that, once all the micro needles have penetrated/punctured the biological membrane, the result will be that at at least one instant of time, they all penetrate/puncture the membrane together.

Preferably, the convexity of the surface 27 is such that the distance between the distalmost tip and the proximalmost tip is at least 50 μm, preferably at least 75 μm, more preferably at least 100 μm in projection on the axis 16.

The maximal distance between the distalmost tip and the proximalmost tip depends on the length of the micro needles 21 and the resilience of the membrane. It should be such that all micro needles can penetrate the membrane to at least a predetermined depth with a single action. Said maximal distance is preferably less than or equal to 2000 μm, preferably less than or equal to 1000 μm, preferably less than or equal to 500 μm, preferably less than or equal to 300 μm.

Preferably, the convexity of the surface 27 is such that all the (finite) radii of curvature of the imaginary surface 27 are located at a same side, i.e. at the side of the micro needles (proximal side).

The indicated arrangement of the micro needles' tips in a convex imaginary surface can be obtained by having the base surface 22 assume the shape of a convex surface. The micro needles 21 can have substantially equal length.

FIG. 3 represents an arrangement in accordance to yet another aspect of the invention, alternative to that of FIG. 2. The base surface 32 of the patch 30 provided with the micro needles 31 is planar and micro needles 31 of different lengths are provided thereon, such that an imaginary surface 37 drawn through the needles' tips 33 is convex as indicated hereinabove.

An advantage of FIG. 3's arrangement is that it is easier to assemble in a liquid distribution unit, as will be evident later. It is also easier and more cost effective to manufacture because of the flat (planar) base surface 32.

Furthermore, FIG. 3's arrangement takes full advantage of the micro needle shape with cavity 34, shell 311 and orifice 35 as brought forward in the present invention. Indeed, since the flow resistance will be mainly determined by the orifice, and the orifice's size is independent of the length of the micro needles, a patch 30 as in FIG. 3 allows obtaining a uniform flow distribution over the micro needles in spite of the differing needle lengths. This would not be the case when a channel were provided through the needles instead of a cavity and orifice.

A combination of non-planar (convex) base surface 27 and micro-needles 31 of different lengths is possible as well. Alternatively, the base surface can be stairway-shaped to avoid the most internal micro needles to become too long (and hence too weak).

It is preferred that the micro needles 11, 21, 31 arranged on a patch 10, 20, 30 are oriented parallel to each other (i.e. that their axes or centre lines are parallel and advantageously parallel to the proximal-distal axis 16).

Patches of micro needles according to the invention are made of a polymeric material, in particular a thermoplastic material. Patches and micro needles made of an injection mouldable material will be most cost effective. Patches may be made according to known manufacturing techniques, such as injection moulding, compression and transfer moulding, thermoforming and deep drawing as e.g. described in U.S. Pat. No. 3,964,482.

Suitable materials for the micro needles are polycarbonate, polystyrene, polyolefins and their variants. Polycarbonate is preferred.

The patches provided with the micro-needles can either be self-supporting, meaning that the base surface has sufficient strength to withstand deformation during penetration of the biological membrane, or not. In the latter case, it needs to be supported such that it can retain its shape during loading. Therefore, a backing layer can be provided at the proximal side of the base surface, to support the base surface and by extension the entire patch.

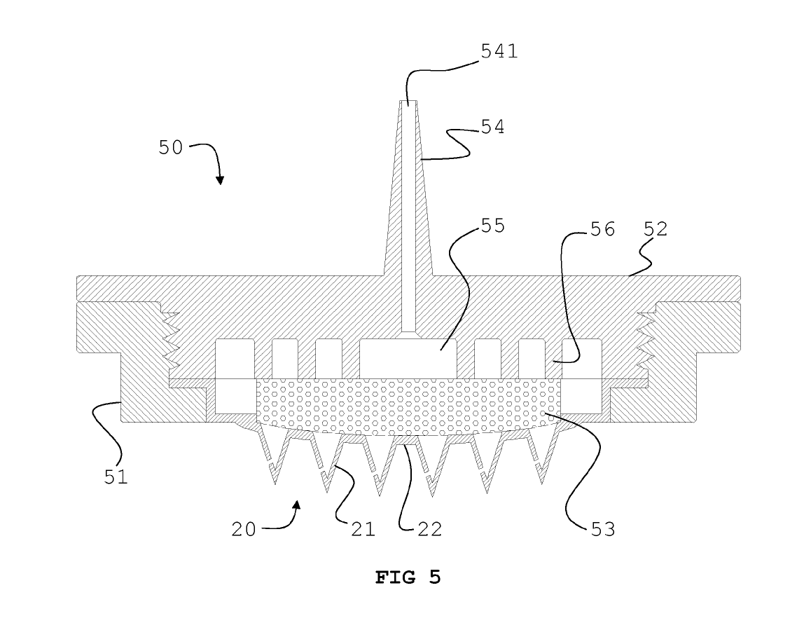

The backing layer is preferably porous. This is represented in FIG. 5, where a porous backing layer 53 is provided at the proximal side of the base surface 22, to support the base surface 22, and by extension the entire patch 20, while allowing the liquid to pass therethrough. FIG. 5 represents a porous backing layer in combination with a patch as in FIG. 2, but it is evident that backing layers can be provided for any patch according to the invention, thus also for the patches 10 and 30 as in FIGS. 1 and 3 respectively.

Patches with micro needles according to the invention can be provided on applicators and may or may not be disposable.

Referring to FIG. 4, any of the patches 10, 20, 30 can be used in injection devices according to the invention. The patch is typically placed in a holder 41 for attaching to a syringe 42 or other supply means of the liquid formulation 43. The combination of holder 41 and patch 10, 20, 30 can be disposable.

In an improvement to devices of FIG. 4, devices of the invention can be arranged to receive a disposable cartridge containing the liquid formulation. Typically, such cartridges will need to be pierced in order to drain the liquid formulation. According to an aspect of the invention, a diffusor unit 50 is provided, as represented in FIG. 5, which serves as interface between the cartridge and the patch 20 of micro needles 21.

At the distal side, diffusor 50 comprises a patch 20 provided with hollow micro needles 21 in accordance with aspects of the invention. The patch 20 is circumferentially housed in a holder 51. Into the holder 51 is screwed a diffusor cap 52, which seals the patch 20 liquid tightly around its circumference.

The diffusor cap 52 is provided with a larger needle 54 at the proximal side. The needle 54 extends from the diffusor cap 52 in proximal direction and is arranged for piercing a cartridge comprising a liquid formulation (not shown). The larger needle 54 is hollow (tubular) and provides a liquid communication path 541 to a liquid distribution manifold 55, which is provided internal in the diffusor 50, between the diffusor cap 52 and the patch 20. The manifold 55 serves to distribute the liquid formulation evenly over the different micro needles 21.

Stalks 56 can be provided within the manifold to support the patch 20. Possibly, a porous backing layer 53 can be provided in addition, or alternative to the stalks 56 for supporting the patch 20. Such a backing layer can be particularly suited for supporting the convex base surface 22.

As an advantage, the diffusor 50 can be provided as a disposable unit, either or not as insert in a screw cap for screwing onto injection devices once a cartridge containing the liquid formulation has been placed. By screwing the screw cap, the tubular needle 54 of the diffusor 50 can be made to pierce the cartridge, so that liquid can be drained and supplied to the micro needles 11 via the distribution manifold 55.

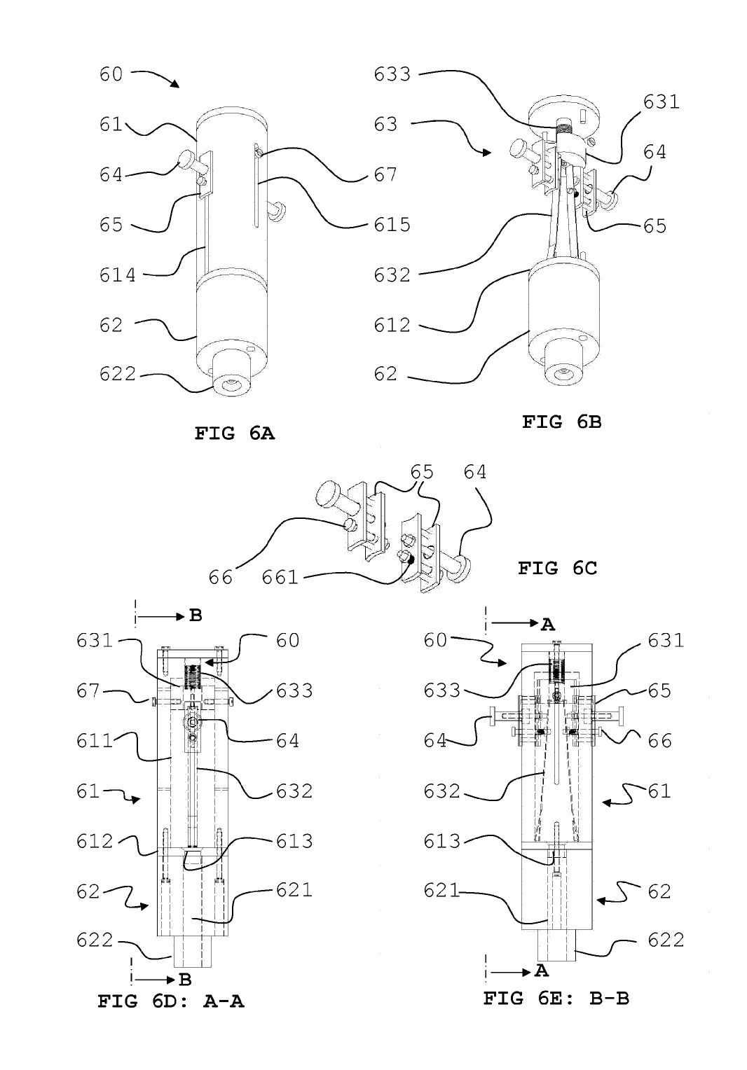

According to another aspect of the invention, an injection device is provided, such as one represented in FIGS. 6A-E. The injection device 60 is not limited to being used with patches of micro needles in accordance to the invention. It can be used with any patch of micro needles. However, use with patches in accordance with aspects of the present invention will provide advantageous effects as indicated.

The injection device 60 comprises an upper part 61 and a lower part 62, both of which may be formed as cylindrical bodies. The lower part 62 extends from the upper part 61 in a distal direction and comprises a seat 621, in the form of a recess, for receiving a cartridge. At the distal end 622 of the lower part 62, means are provided for attaching a patch of micro needles. By way of example, the lower part 62 can be provided at the distal end 622 with external thread for screwing a cap thereon. The cap can comprise the patch of micro needles.

Preferably, a cap for attaching to the distal end 622 of the injection device 60 can comprise a diffusor unit provided with a tubular needle for piercing the cartridge, such as the one indicated above with reference to FIG. 5. By attaching (e.g. by screwing) the cap onto the lower part of the injection device 60, the cartridge will be pierced and liquid can be drained therefrom.

The upper part 61 of the injection device 60 comprises means 63 for draining the cartridge seated in the lower part 62. Preferably, such means comprise a member 631 moveably arranged in a shaft (passageway) 611 internal to the upper part 61 and at least one stem 632 attached thereto so as to move together with the member 631. The member 631 is spring-loaded by a spring 633 to move the member 631 and the stem 632 along shaft 611 in the direction of the cartridge (seat 621). The stem 632 is biased so as to assume an orientation transverse or oblique to the axis of shaft 611.

In the example of FIGS. 6A-E, two such stems 632 are provided, attached to the member 631 at the distal side thereof (i.e. at the side towards the cartridge) for moving together with the member 631. The stems 632 are biased (e.g. spring-loaded) so that their distal ends point away from each other. The stems 632 adopt the shape of an inverted V (see FIGS. 6B, E).

At the distal side, the upper part 61 comprises retaining means for stems 632, such as a platform 612, onto which the stems' 632 distal ends are arranged to rest. When the stems 632 rest on the platform 612, the spring 633 is loaded (compressed) and the member 631 is maintained in a proximal position. The platform 612 is provided with a through-hole 613, in line with the stems 632 and providing a passageway to the cartridge's seat 621. Through-hole 613 is provided in between the rest positions of the stems 632 on platform 612 and has a size to let the stems 632 pass through.

Push buttons 64, provided through longitudinal slits 614 along the upper part's body 61 are arranged to contact the stems 632 for displacing them against the biasing force. By so doing, the stems' distal ends will eventually leave the platform 612 to end up in front of the through-hole 613. In the latter position, the stems 632 are oriented in line with the through-hole 613 and can thus pass through. In this position, the spring 631 will be able to unload and move the member 631 and the stems 632 in distal direction (towards the cartridge).

A slide 65 can be provided, arranged for sliding in a longitudinal slit 614, which is oriented parallel to the direction of motion of the member 631.

Slide 65 (and slit 614) provide access to the shaft 614 in which stems 632 are provided. Slide 65 is preferably arranged to move independently of the combination of member 631 and stems 632. Push buttons 64 may or may not be attached to the slide 65 and may or may not be attached to stems 632.

The injection device 60 preferably further comprises engaging means, arranged for engaging the stem 632. The engaging means can be formed of a pin-like member 66, arranged for engaging a recess or hole of the stem 632. The member 66 is arranged transversally to the stem's orientation and is biased (e.g. spring loaded by a spring 661) towards the stem 632.

The stem engaging means' purpose is to move the stems 632 (and also the member 631) back to the rest position wherein the spring 633 is loaded and the stems 632 rest on the platform 612.

The stem engaging means 66 may be provided on the slide 65. The stem engaging means 66 is preferably releasable from the stem. This will allow the slide 65 to move independently of the member-and-stems combination 631-632. Alternatively, the stem engaging means 66 and possibly the slide 65 may be arranged to move together with the member-and-stems combination 631-632.

The injection device may comprise a level indicator, preferably formed of a pin 67 attached to the member 631 along a transverse direction and extending out of the upper part's body 61 through a longitudinal slit 615. The level indicator 67 is particularly of use in case the stem engaging means 66 and the slide 65 and push button 64 are not arranged to move together with the member-and-stems combination 631-632, so as to provide a user of the injection device 60 with an indication of the position of the member 631.

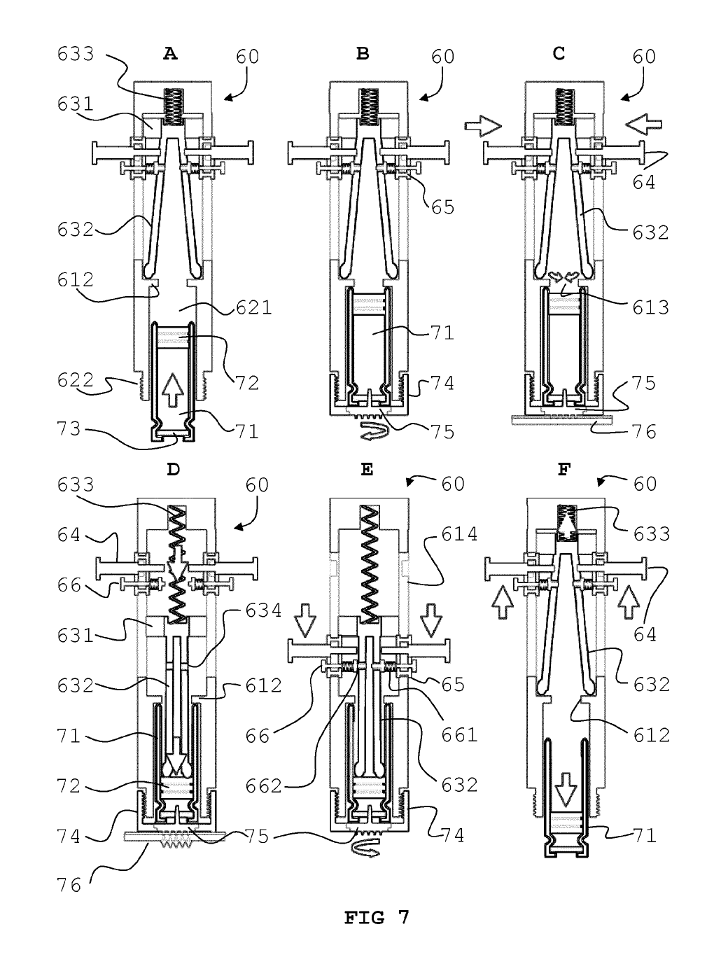

The use of the injection device 60 is now explained by referring to FIG. 7. In a first step A, the device 60 is in a rest position, with member 631 and stems 632 retracted. The stems' distal ends rest on platform 612, which keeps the spring 633 loaded. In this position of the injection device, a cartridge 71 can be inserted in the seat 621 through a distal opening 622.

Cartridge 71 comprises the liquid formulation to be injected. It is preferably of a type comprising a movable bottom wall 72 at one end and a pierceable closing membrane 73 at the opposite end. Cartridge 71 is inserted with its bottom wall 72 directed towards the stems 632.

In a following step B, a cap 74 comprising the micro needles is screwed onto the injection device at the distal end 622 to close the seat 621. Cap 74 comprises a diffusor 75 provided with a patch of (hollow) micro needles at one end and with a tubular needle at the other end. The tubular needle pierces the cartridge's closing membrane 73 when the cap is screwed. Diffusor 75 comprises an internal distribution manifold which enables distributing the liquid formulation over the micro needles.

The injection device is now ready for being used. In a subsequent step C, the injection device is applied onto a target membrane 76 (e.g. the skin), so that the micro needles puncture it and possibly penetrate thereinto. In order to prevent spilling of the liquid formulation, all micro needles (in fluid communication with the cartridge 71) should puncture/penetrate the skin 76.

Once the micro needles are in target position, an operator/user can commence injecting the liquid formulation. To do so, the operator presses push buttons 64 in the direction indicated by the arrows. The push buttons 64 hence press the two stems 632 against their biasing force (i.e. towards each other) until these are released from the platform 612 and face the through-hole 613. By so doing, the stem engaging means 66 are also released from the stems 632. The stems 632 and member 631 are now free to move. This action starts the automatic draining of the cartridge 71 as is explained with reference to step D.

In step D, once the stems 632 have left the platform, the loaded spring 633 will force the stems 632 to move through the hole 613 to contact the cartridge's bottom wall 72. The spring 633 will then continue to exert a force on the stems 632 to move the bottom wall 71 towards the cartridge's opposite end and thereby drain the liquid formulation from the cartridge. The drained liquid formulation flows to diffusor 75, from where it is injected into the skin 76.

The push buttons 64 are preferably not attached to the stems 632, and preferably allow the stems 632 and the attached member 631 to move along while the push buttons 64 remain in position. This has the advantage that the operator does not need to take care of releasing or freeing the push buttons immediately after the stems 632 have been moved towards a working position (step C). Also, the injection/draining of liquid formulation is effected automatically, without the risk of inadvertent blocking by the operator (which would be the case when the operator is carelessly retaining the stems and the push buttons were blocking passage of the member 631).

Once the cartridge is emptied, the injection device is removed from the skin. The cap 74 is screwed off and may be disposed of. In order to remove the empty cartridge, the stems have to be retracted first. This is performed as shown in step E. A slide 65, moveable in a slit 614, is provided with stem engaging means 66 arranged to engage with the stem 632, such as by locking into a recess 634 of the stem 632. Recess 634 can be a hole or hook in the stem. The stem engaging means 66 comprises an engagement pin 662 which is biased towards the stem 632, e.g. by a spring 661.

The slide 65 is moved downwards (towards the cartridge) along slit 614 as indicated by the arrows in step E, until the engagement pin 662 locks into the recess 634. The slide 65 is now attached to the stem 63. Moving the slide 65 upwards as indicated by the arrows in step F allows to retract the stems 632 out of the cartridge and to put them back into the rest position as indicated in step A. At the same time, the spring 633 is loaded for subsequent use. In the latter position, the empty cartridge 71 can be removed from the device 60.

Manual operation of the slide 65 is eased when the push button 64 is attached to the slide 65, so that it can be used as a handle.

Injection devices according to the invention have been described with two stems 632. It is however to be noted that same operation can be obtained with one (biased) stem, or more than two stems.

Even though injection devices have been described with a push button and engaging means for each stem, they can be construed with a single push button and engaging means (to engage one or more stems) without loss of functionality.

Optionally, a skin tensioning means, as e.g. known from US 2008/183144, can be provided on injection device 60.

It is finally to be noticed that injection devices according to the invention can be used with all kinds of patches with hollow micro needles. They can be used with a single injection needle instead of a patch of micro needles as well.

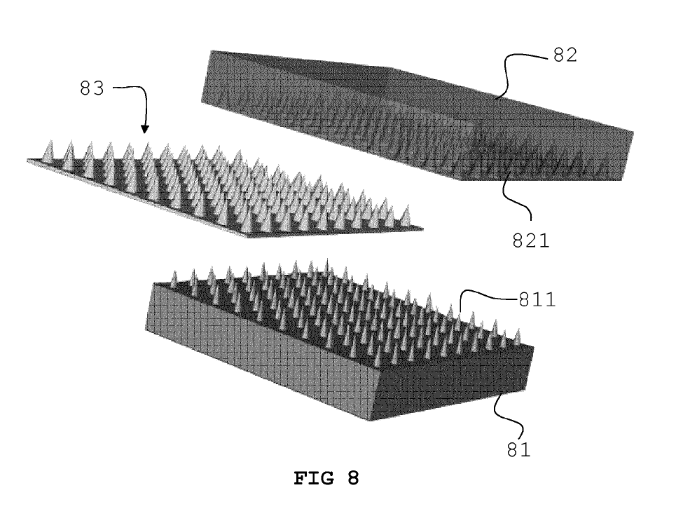

In accordance with an aspect of the invention, there is provided a process for manufacturing patches of micro needles, such as the ones contemplated by the present invention. The manufacturing process is advantageously an injection moulding process, in which an injection mouldable material is injected into a mould, which, as shown in FIG. 8, basically comprises two inserts, a male insert 81 and a female insert 82, defining respectively the proximal and distal surfaces of the patch of micro needles 83.

The male insert 81 comprises projections 811, which correspond to the cavities of the micro needles. It can be made of metal and can be formed by appropriate techniques, such as micro-machining. As the cavities have preferably conical shape, the projections 811 will have such shape as well.

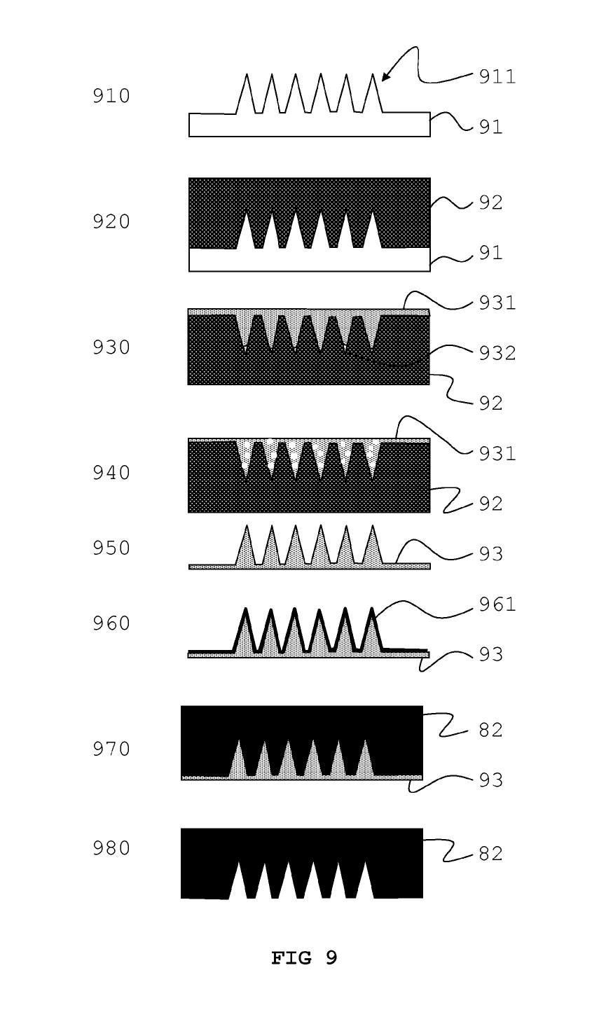

The female insert 82 comprises recesses 821 corresponding to the outer shape of the micro needles. The dimensions and shape (e.g. sharp tip) of the recesses make the fabrication of the female insert 82 a challenging task. The female insert 82 can advantageously be made through a duplication technique as will be explained by reference to FIG. 9.

At first, in a step 910, a negative 91 of the female insert 82 is manufactured, e.g. by micro-machining. The upper surface 911 of the negative 91 corresponds to the outer shape of the micro needles, and hence to the inner mould surface (side of the recesses) of the female insert 82.

The negative 91 is duplicated in a polymeric material, such as an epoxy material. Duplication can be effected as follows. An elastomeric material, such as silicone, is moulded around the negative 92, to obtain a positive imprint 92 of the female part in step 920. The positive imprint 92 is demoulded from the negative 91 and filled (at the side of the recesses) with a (liquid) polymeric material 931, such as epoxy, in a step 930. The polymeric material 931 can be suitably conditioned, e.g. by degassing, in order to avoid any inclusions 932 of air, in particular at the sharp valley of the recesses in a step 940. After curing and demoulding, a duplicate 93 of the negative 91 is obtained in a step 950.

The actual female part 82 is then fabricated based on the duplicate 93, preferably by an electrodeposition process. Such process has the advantage that it can very accurately replicate the outer surface of the duplicate 93 as internal surface of an insert. A preferred electrodeposition process is electroforming, such as with nickel. By way of example, the duplicate 93 is first covered with a thin metal layer 961 by a metal deposition technique in a step 960. Subsequently, the metal layer 961 is further covered with a metal by way of an electroforming process to form the female part 82 in a step 970. The duplicate 93 can easily be removed, e.g. by dissolution to obtain the female part 82 in a step 980.

The two inserts 81 and 82 are then assembled in an injection moulding device to manufacture the patches of micro needles by injection moulding.

The injection moulding process can be suitably conditioned in order to ensure that air entrapment during moulding is avoided. Possibly, injection moulding is performed under a vacuum to avoid air entrapment and ensure that the tips of the micro needles are moulded as desired.

To obtain micro needles 11, 21, 31 according to aspects of the invention, the projections 811 of the male mould insert 81 and the recesses 821 of the female mould insert 82 are correspondingly shaped, such that when the inserts are assembled, a substantially constant clearance is obtained between each recess 821 and corresponding projection 811, at least between the base surface of the recesses and the tips of the projections.

The assembled injection mould with inserts 81 and 82 hence allows for obtaining an injection moulded patch of hollow but blind micro needles. This means that the micro needles as obtained through the injection moulding process do comprise a virgin, intact shell without any orifices or outlets (towards the distal side). Hence, by injection moulding a non-porous (or liquid-tight) patch of micro needles is obtained. The micro needles' cavities 14, 24, 34 as obtained with injection moulding therefore do not comprise other openings than towards the proximal side.

The orifices and channels are formed only afterwards, in a step following demoulding the patch of blind micro needles from the injection mould. The orifices and channels are formed in this subsequent step by piercing the shell, such as by ablation with a laser beam, preferably an excimer laser.

When forming the orifices and channels through laser ablation or any other technique, the laser beam or other piercing means is preferably made incident on the micro needle's shell under an inclination relative to the proximal-distal axis, with advantageous inclination angles as defined above in relation to FIG. 11 (angle β).

An advantage of forming channels and orifices offset from the tip and under an inclination relative to the micro needle's axis, is that any operation to open an outlet or orifice spares the micro needle tip. By way of example, in case of laser ablation, the heat that is introduced in the micro needle by the laser radiation, will cause no risk of deformation of the tip. This is e.g. not the case in DE 10 2008 052 749, where the orifice and channel are very close to the tip and have same orientation.

A further advantage of manufacturing micro needles with shells of substantially constant thickness is that the positioning accuracy requirements of e.g. a laser beam for opening the channels can be relaxed, since even in case of a slight positioning error, it is always ensured that the channel is pierced completely through the shell. This would not be the case when the shell's thickness would not be constant.

Yet another advantage of methods according to the invention, is that the moulds are kept as simple as possible and no additional, slender protrusions need be provided. Furthermore, any mating contact between the projections and recesses is avoided, so that the inserts can have longer lifetime than inserts for current hollow micro-needles.

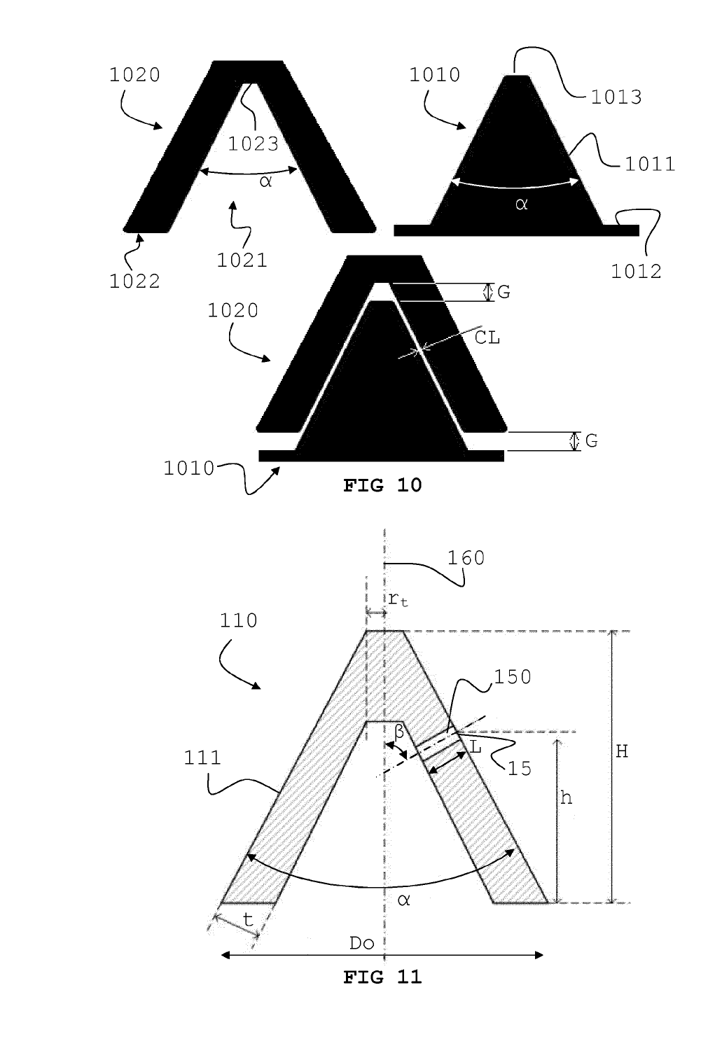

In an advantageous embodiment, the projections 811 of the male mould insert 81 and the recesses 821 of the female mould insert 82 have identical shape and dimensions, i.e. they are obtained by a same master. Referring to FIG. 10, recesses 1021 of the female mould insert 1020 are identical negative imprints of projections 1011 of male mould insert 1010. That is, the two male mould insert 1010 can be inserted completely in the female mould insert 1020 to obtain a full match, without any gaps, between projections 1011 and recesses 1020. It will be convenient to note that there may be a gap between respective base surfaces 1012 and 1022 of the male and female mould inserts (not shown in FIG. 10).

When assembling the mould, it will suffice to arrange the two mould inserts at a distance G from each other so as to obtain a clearance CL between the walls of recesses 1021 and projections 1011 which is substantially constant. The clearance G between the tips 1013 and 1023 of the male respectively female inserts 1010, 1020 and between the base planes 1012 and 1022 of the male respectively the female insert can differ from CL. As is the case in the example of FIG. 10, G is larger than CL: CL=G sin(α/2), with a the cone angle. Since only one master is required to manufacture the mould inserts, the mould manufacturing costs can be substantially reduced. By way of example, and referring to FIG. 9, a male mould insert 91 can be used as master and a female mould insert 82 can be produced by method steps 910-980 as explained above.

The base surfaces of the inserts 81, 82 are preferably planar, or stairway-stepped.

The tips of the male insert's projections and the “valleys” of the female insert's recesses can be suitably arranged in convex/concave surfaces to provide patches of micro needles as described above in accordance with aspects of the invention.

1.-26. (canceled)

27. A device for puncturing a membrane of a human or animal body so as to introduce a liquid formulation thereinto, comprising a base surface having a distal side and a proximal side, and a plurality of projections, each projection projecting from the distal side of the base surface to end in a tip arranged for puncturing the membrane;
wherein each projection comprises a cavity having a cross sectional size which varies along a proximal-distal axis and a shell enveloping the cavity at the distal side, wherein the cavity is open towards the proximal side of the base surface, wherein the shells and the base surface are made of a thermoplastic material, wherein the shells have substantially constant thicknesses between the base surface and the tips, and wherein an orifice for passage of the liquid formulation from the cavity to the distal side is provided in the shell offset from the tip, wherein the projections comprise fluid channels emanating from the cavities, running through the shells to end at the orifices, the fluid channels being oriented obliquely relative to the proximal-distal axis, and wherein the shells, disregarding the orifices and the fluid channels, are axisymmetric about the proximal-distal axis.

28. The device of claim 27, wherein the projections have a length falling in the range between 100 μm and 1000 μm and the cavity extends along at least 50% of said length.

29. The device of claim 27, wherein the shells have a thickness in the range between 150 μm and 10 μm.

30. The device of claim 27, wherein the fluid channels are oriented such that the fluid channels make an angle with the proximal-distal axis of at least 15° from the distal side.

31. The device of claim 30, wherein the angle is at least 25°.

32. The device of claim 30, wherein the angle is smaller than or equal to 80°.

33. The device of claim 27, wherein the cavity has essentially the shape of a right circular cone.

34. The device of claim 27, wherein the tip has a radius of curvature between 10 μm and 50 μm.

35. The device of claim 27, wherein the cavity has a size at the base surface between 80 μm and 950 μm and the orifice has a size smaller than 0.5 times the cavity's size.

36. The device of claim 27, wherein the cavity has a size at the base surface between 80 μm and 950 μm and the orifice has a size falling in the range between 0.1 and 0.5 times the cavity's size.

37. The device of claim 35, wherein the cavity has a size at the base surface between 80 μm and 450 μm.

38. The device of claim 27, wherein the tips of the projections are arranged such that a surface which is drawn through the tips is convex along at least one direction when considered from the distal side.

39. The device of claim 38, wherein the distance between the most distal tip and the most proximal tip is at least 50 μm in projection normal to the base surface.

40. device of claim 38, wherein the base surface is substantially planar and wherein the projections have differing lengths from base surface to tip so as to obtain the convex surface.

41. The device of claim 27, wherein the thermoplastic material is polycarbonate.

42. The device of claim 27, comprising a distribution manifold arranged at the proximal side of the base surface and in liquid communication with the cavities, and a tubular needle in liquid communication with the manifold, wherein the manifold is interposed between the tubular needle and the base surface and the tubular needle projects from the manifold in a proximal direction, wherein the tubular needle has a shape and size configured for piercing a container containing the liquid formulation and for draining the liquid formulation therefrom.

43. A disposable unit comprising the device of claim 42, and being configured for attachment to an injection device releasably accepting the container, the injection device being operable to drain the container.

44. A kit of parts operable to administer a liquid formulation into a human or animal body, the kit comprising the disposable unit of claim 43 and an injection device comprising a body having an open-ended recess releasably accepting a container containing the liquid formulation, the body and the disposable unit being configured to be removably secured to each other at the recess' open end, the injection device further comprising means for draining the container.

45. A method of manufacturing a patch of hollow, substantially conically shaped projections for puncturing a membrane of a human or animal body so as to introduce a liquid formulation thereinto, comprising:
providing an injection mould having a male insert and a female insert in facing relationship, wherein the male insert is provided with a first base surface and projections of varying cross sectional shape projecting from the base surface towards a tip, and the female insert is provided with a second base surface and blind recesses forming depressions in the second base surface, the recesses being disposed at locations corresponding to the projections, wherein the projections and the recesses are shaped such that when the injection mould is assembled, a substantially constant clearance between each projection and corresponding recess is obtained, at least between the second base surface and the tip, wherein the clearance is axisymmetric about a proximal-distal axis oriented from the second base surface to the tip;
injecting a thermoplastic material in the injection mould to form a patch of blind micro needles provided with cavities formed by the projections extending between a base surface and tips and with shells wrapping the cavities; and
removing the patch from the injection mould followed by forming channels through the shells at positions offset from the micro needles' tips and under orientations oblique to the proximal-distal axis.

46. The method of claim 45, wherein the channels are formed by laser ablation.

47. The device of claim 27, wherein the projections have a length falling in the range between 100 μm and 1000 μm and the cavity extends along at least 60% of said length.
